(19) United States
(12) Patent Application Publication
(10) Pub. No.: US 2002/0049311 A1
(43) Pub. Date: Apr. 25, 2002

(54) NOVEL RNASE P

(76) Inventors: MICHAEL J GRESS, WAYNE, PA (US); LISA A HEGG, DEVON, PA (US); HU LI, COLLEGEVILLE, PA (US); CATHERINE D PRESCOTT, CAMBRIDGE (GB); AMY M SHAPPELL, SHOEMAKERSVILLE, PA (US)

Correspondence Address: DECHERT, ATTN: ALLEN BLOOM, ESQ, 4000 BELL ATLANTIC TOWER, 1717 ARCH STREET, PHILADELPHIA, PA, 19103, US

(21) Appl. No.: 09/214,474

(22) PCT Filed: Sep. 3, 1998
(86) PCT No.: PCT/US98/18291

(51) Int. Cl. C12N 15/00

(52) U.S. Cl. 536/23.2; 435/183; 435/115; 435/320.1; 435/325

(57) ABSTRACT

This invention relates to a novel bacterial ribonucleoprotein complex and the component parts thereof More specifically, this invention relates to RNase P isolated from S. pneumoniae and the use of RNase P or components thereof in screens for the identification of antimicrobial compounds and to the use of such compounds in therapy.

### FIELD OF THE INVENTION

This invention relates to newly identified polynucleotides, polypeptides and catalytic RNAs encoded by certain of these polynucleotides, molecular complexes of RNAs and polypeptides, uses of such polynucleotides and polypeptides, as well as the production of such polynucleotides and polypeptides and recombinant host cells transformed with the polynucleotides. The invention relates particularly to such polynucleotides and polypeptides from Streptococci, especially Streptococcus pneumoniae (S. pneumoniae). This invention also relates to inhibiting the biosynthesis, assembly or action of such polynucleotides and/or polypeptides and to the use of such inhibitors in therapy.

### BACKGROUND OF THE INVENTION

This invention relates to a novel bacterial ribonucleoprotein complex and the component parts thereof. More specifically, this invention relates to RNase P, particularly RNase P from S. pneumoniae, and the use of RNase P or components thereof in screens for the identification of antimicrobial compounds and to the use of such compounds in therapy.

The Streptococci make up a medically important genera of microbes known to cause several types of disease in humans, including, for example, otitis media, conjunctivitis, pneumonia, bacteremia, meningitis, sinusitis, pleural empyema and endocarditis, and most particularly meningitis, such as for example infection of cerebrospinal fluid. Since its isolation more than 100 years ago, Streptococcus pneumoniae has been one of the more intensively studied microbes. For example, much of our early understanding that DNA is, in fact, the genetic material was predicated on the work of Griffith and of Avery, Macleod and McCarty using this microbe. Despite the vast amount of research with S. pneumoniae many questions concerning the virulence of this microbe remain. It is particularly preferred to employ Streptococcal genes and gene products as targets for the development of antibiotics.

The frequency of Streptococcus pneumoniae infections has risen dramatically in the past 20 years. This has been attributed to the emergence of multiply antibiotic resistant strains and an increasing population of people with weakened immune systems. It is no longer uncommon to isolate Streptococcus pneumoniae strains which are resistant to some or all of the standard antibiotics. This has created a demand for both new anti-microbial agents and diagnostic tests for this organism.

While certain Streptococcal proteins associated with pathogenicity have been identified, additional targets are always useful because it is appreciated that the target of a antimicrobial screen can often bias the outcome. Thus, new targets such as RNaseP of the invention allow for the discovery of new classes of antimicrobials.

### BRIEF DESCRIPTION OF THE INVENTION

This invention provides a novel ribonucleoprotein complex, particularly such complex from S. pneumoniae, and the separately isolated RNA and protein components thereof.

In accordance with another aspect of the present invention, there are provided polynucleotides (DNA or RNA) which encode the protein and RNA components of such a complex.

In particular the invention provides polynucleotides having the DNA and RNA sequences given herein.

The invention also relates to novel oligonucleotides derived from the sequences given herein which can act, for example, as antisense inhibitors of the expression of the RNA or protein components. The oligonucleotides or fragments or derivatives thereof can also be used to directly inhibit catalytic activity or indirectly inhibit activity by interference with RNA protein complex formation. The protein and the RNA components, either separately or in a complex, are also useful as targets in screens designed to identify antimicrobial compounds.

It is an object of the invention to provide polynucleotides that are transcribed into RNase P RNA, particularly catalytic RNA.

A further object of the invention is to provide a polynucleotide isolated from pneumoniae using the nucleic acid sequence set forth in SEQ ID NO: 1 or 5 as a probe or primer.

A still further object of the invention is to provide a polynucleotide isolated from pneumoniae set forth in SEQ ID NO:5, and variants thereof.

In accordance with another aspect of the invention there is provided an isolated nucleic acid molecule that is transcribed into an RNase P RNA by the S. pneumoniae strain 0100993 contained in the deposited strain.

A further aspect of the invention there are provided isolated nucleic acid molecules encoding RNaseP RNA, particularly S. pneumoniae RNaseP, including mRNAs, cDNAs, genomic DNAs and catalytic RNAs. Further embodiments of the invention include biologically, diagnostically, prophylactically, clinically or therapeutically useful variants thereof, and compositions comprising the same.

In accordance with another aspect of the invention, there is provided the use of a polynucleotide of the invention, particularly those encoding polypeptide, for therapeutic or prophylactic purposes, in particular genetic immunization. Among the particularly preferred embodiments of the invention are naturally occurring allelic variants of RNaseP and polypeptides encoded thereby.

As another aspect of the invention there are provided novel polypeptides of S. pneumoniae referred to herein as RNaseP as well as biologically, diagnostically, prophylactically, clinically or therapeutically useful variants thereof, and compositions comprising the same.

Among the particularly preferred embodiments of the invention are variants of RNaseP polypeptide encoded by naturally occurring alleles of the RNaseP gene.

In a preferred embodiment of the invention there are provided methods for producing the aforementioned RNaseP polypeptides.

In accordance with yet another aspect of the invention, there are provided inhibitors to such polypeptides, useful as antibacterial agents, including, for example, antibodies.

In accordance with certain preferred embodiments of the invention, there are provided products, compositions and methods for assessing RNaseP expression, treating disease, for example, otitis media, conjunctivitis, pneumonia, bacteremia, meningitis, sinusitis, pleural empyema and endocarditis, and most particularly meningitis, such as for example infection of cerebrospinal fluid, assaying genetic variation, and administering a RNaseP polypeptide or polynucleotide to an organism to raise an immunological response against a bacteria, especially a S. pneumoniae bacteria.

In accordance with certain preferred embodiments of this and other aspects of the invention there are provided polynucleotides that hybridize to RNaseP polynucleotide sequences, particularly under stringent conditions.

In certain preferred embodiments of the invention there are provided antibodies against RNaseP polypeptides.

In other embodiments of the invention there are provided methods for identifying compounds which bind to or otherwise interact with and inhibit or activate an activity of a polypeptide or polynucleotide of the invention comprising: contacting a polypeptide or polynucleotide of the invention with a compound to be screened under conditions to permit binding to or other interaction between the compound and the polypeptide or polynucleotide to assess the binding to or other interaction with the compound, such binding or interaction being associated with a second component capable of providing a detectable signal in response to the binding or interaction of the polypeptide or polynucleotide with the compound; and determining whether the compound binds to or otherwise interacts with and activates or inhibits an activity of the polypeptide or polynucleotide by detecting the presence or absence of a signal generated from the binding or interaction of the compound with the polypeptide or polynucleotide.

In accordance with yet another aspect of the invention, there are provided RNaseP agonists and antagonists, preferably bacteriostatic or bacteriocidal agonists and antagonists.

In a further aspect of the invention there are provided compositions comprising a RNaseP polynucleotide or a RNaseP polypeptide for administration to a cell or to a multicellular organism.

Various changes and modifications within the spirit and scope of the disclosed invention will become readily apparent to those skilled in the art from reading the following descriptions and from reading the other parts of the present disclosure.

### DETAILED DESCRIPTION OF THE FIGURES

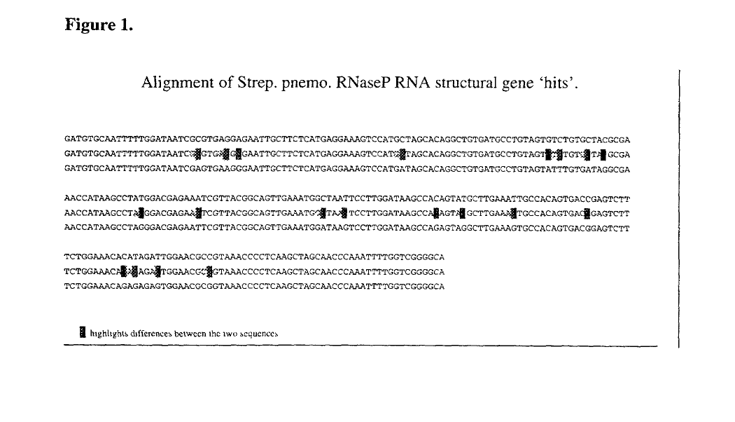

FIG. 1 illustrates an alignment of the two sequence in FIG. 1 against each other with the differences highlighted in gray. There are 20 differences over the first 280 nucleotides.

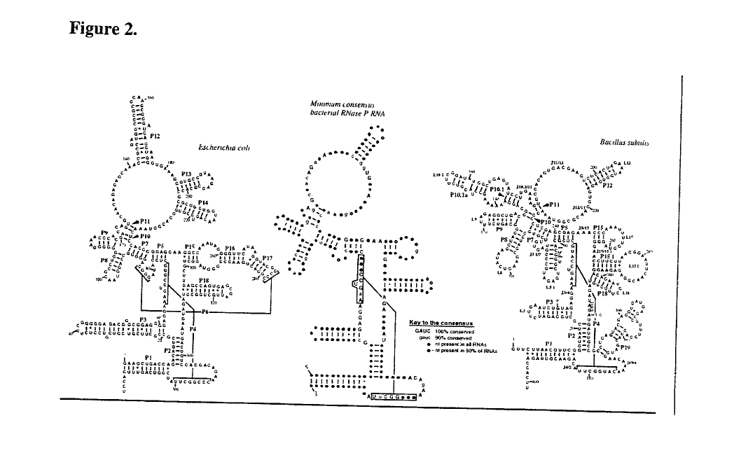

FIG. 2 illustrates secondary structure predictions of the E.coli and B. subtilis RNaseP. A minimum consensus RNase P RNA structure is also illustrated.

Glossary:

The following definitions are provided to facilitate understanding of certain terms used frequently herein. Certain other definitions are provided elsewhere herein.

“Host cell” is a cell which has been transformed or transfected, or is capable of transformation or transfection by an exogenous polynucleotide sequence.

“Identity,” as known in the art, is a relationship between two or more polypeptide sequences or two or more polynucleotide sequences, as the case may be, as determined by comparing the sequences. In the art, “identity” also means the degree of sequence relatedness between polypeptide or polynucleotide sequences, as the case may be, as determined by the match between strings of such sequences. “Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following: Algorithm: Needleman and Wunsch, J. Mol Biol. 48: 443-453 (1970) Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis.. The aforementioned parameters are the default parameters for peptide comparisons (along with no penalty for end gaps).

Parameters for polynucleotide comparison include the following: Algorithm: Needleman and Wunsch, J. Mol Biol. 48: 443453 (1970)

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

A preferred meaning for “identity” for polynucleotides and polypeptides, as the case may be, are provided in (1) and (2) below.

(1) Polynucleotide embodiments further include an isolated polynucleotide comprising a polynucleotide sequence having at least a 50, 60, 70, 80, 85, 90, 95, 97 or 100% identity to the reference sequence of SEQ ID NO: 1, 3, 4 or 7, wherein said polynucleotide sequence may be identical to the reference sequence of SEQ ID NO: 1, 3, 4 or 7, or may include up to a certain integer number of nucleotide alterations as compared to the reference sequence, wherein said alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein said number of nucleotide alterations is determined by multiplying the total number of nucleotides in SEQ ID NO: 1, 3, 4 or 7, by the integer defining the percent identity divided by 100 and then subtracting that product from said total number of nucleotides in SEQ ID NO: 1, 3, 4 or 7, or:

nₙ≦xₙ−(xₙ·y),

wherein nₙ is the number of nucleotide alterations, xₙ is the total number of nucleotides in SEQ ID NO: 1, 3, 4 or 7, y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and · is the symbol for the multiplication operator, and wherein any non-integer product of xₙ and y is rounded down to the nearest integer prior to subtracting it from xₙ. Alterations of a polynucleotide sequence encoding the polypeptide of SEQ ID NO:2 or 6, may create nonsense, missense or frameshift mutations in this coding sequence and thereby alter the polypeptide encoded by the polynucleotide following such alterations.

By way of example, a polynucleotide sequence of the present invention may be identical to the reference sequence of SEQ ID NO: 1, 3, 4 or 7, that is it may be 100% identical, or it may include up to a certain integer number of nucleic acid alterations as compared to the reference sequence such that the percent identity is less than 100% identity. Such alterations are selected from the group consisting of at least one nucleic acid deletion, substitution, including transition and transversion, or insertion, and wherein said alterations may occur at the 5′ or 3′ terminal positions of the reference polynucleotide sequence or anywhere between those terminal positions, interspersed either individually among the nucleic acids in the reference sequence or in one or more contiguous groups within the reference sequence. The number of nucleic acid alterations for a given percent identity is determined by multiplying the total number of nucleic acids in SEQ ID NO:1, 3, 4 or 7 by the integer defining the percent identity divided by 100 and then subtracting that product from said total number of nucleic acids in SEQ ID NO: 1 or 5, or:

nₙ≦xₙ−(xₙ·y),

wherein nₙ is the number of nucleic acid alterations, xₙ is the total number of nucleic acids in SEQ ID NO: 1, 3, 4 or 7, y is, for instance 0.70 for 70%, 0.80 for 80%, 0.85 for 85% etc., · is the symbol for the multiplication operator, and wherein any non-integer product of xₙ and y is rounded down to the nearest integer prior to subtracting it from xₙ.

(2) Polypeptide embodiments further include an isolated polypeptide comprising a polypeptide having at least a 50, 60, 70, 80, 85, 90, 95, 97 or 100% identity to a polypeptide reference sequence of SEQ ID NO:2 or 6, wherein said polypeptide sequence may be identical to the reference sequence of SEQ ID NO:2 or 6, or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in SEQ ID NO:2 or 6, by the integer defining the percent identity divided by 100 and then subtracting that product from said total number of amino acids in SEQ ID NO:2 or 6, or:

nₐ≦xₐ−(xₐ·y),

wherein nₐ is the number of amino acid alterations, xₐ is the total number of amino acids in SEQ ID NO:2 or 6, y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and · is the symbol for the multiplication operator, and wherein any non-integer product of xₐ and y is rounded down to the nearest integer prior to subtracting it from xₐ.

By way of example, a polypeptide sequence of the present invention may be identical to the reference sequence of SEQ ID NO:2 or 6, that is it may be 100% identical, or it may include up to a certain integer number of amino acid alterations as compared to the reference sequence such that the percent identity is less than 100% identity. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:2 or 6, by the integer defining the percent identity divided by 100 and then subtracting that product from said total number of amino acids in SEQ ID NO:2 or 6, or:

nₐ≦xₐ−(xₐ·y),

wherein nₐ is the number of amino acid alterations, xₐ is the total number of amino acids in SEQ ID NO:2 or 6, y is, for instance 0.70 for 70%, 0.80 for 80%, 0.85 for 85% etc., and · is the symbol for the multiplication operator, and wherein any non-integer product of xₐ and y is rounded down to the nearest integer prior to subtracting it from xₐ.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein.

“Bacteria(l)” means a (i) prokaryote, including but not limited to, a bacterial host cell, such as a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturelia, Moraxella, Acinetobacter, Erysipelothrix, Branhamella, Actinobacilluts, Streptobacillus, Listeria, Calymmatobacteriutn, Brucela, Bacillus, Clostridium, Treponema, Escherichia, Salmonella, Kleibsiella, Vibrio, Proteus, Erwinia, Borrelia, Leptospira, Spirillur, Campylobacter, Shigella, Legionella, Pseudomonas, Aeromonas, Rickettsia, Chlamydia, Borrelia and Mycoplasma, and further including, but not limited to, a member of the species or group, Group A Streptococcus. Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus ditrans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacteriurm leprae, Actinomyctes israelii, Listeria monocytogenes, Bordetella pertusis, Bordatella parapertusis, Bordetella bronchiseptica, Escherichia coli, Shigella dysenteriae, Haemophilus influenizae, Haemophilus aegyptius, Haemophilus parainfluenzae, Haemophilus ditcreyi, Bordetella, Salmonella typhi, Citrobactet freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostriditm perfringens, Clostridium tetani, Clostridium botulinum, Treponema pallidum, Rickettsia rickettsii and Chlamydia trachomitis, and (ii) an archaeon, including but not limited to Archaebacter.

“Polynucleotide(s)” generally refers to any polyribonucleotide or polydeoxribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini, Modifications include, for example, acetylation, acylation, ADP-ribosylation, amidation, covalent attachment of flavin. covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation. formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues. hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

“Plasmids” are designated by a lower case p preceded and/or followed by capital letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

“Digestion” of DNA refers to catalytic cleavage of the DNA with a restriction enzyme that acts only at certain sequences in the DNA. The various restriction enzymes used herein are commercially available and their reaction conditions, cofactors and other requirements were used as would be known to the ordinarily skilled artisan. For analytical purposes, typically 1 μg of plasmid or DNA fragment is used with about 2 units of enzyme in about 20 μl of buffer solution. For the purpose of isolating DNA fragments for plasmid construction, typically 5 to 50 μg of DNA are digested with 20 to 250 units of enzyme in a larger volume. Appropriate buffers and substrate amounts for particular restriction enzymes are specified by the manufacturer. Incubation times of about 1 hour at 37° C. are ordinarily used, but may vary in accordance with the supplier's instructions. After digestion the reaction is electrophoresed directly on an agarose gel to isolate the desired fragment. Size separation of the cleaved fragments is generally performed using a 1% agarose gel.

“Oligonucleotides” refers to either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

“Ligation” refers to the process of forming phosphodiester bonds between two double stranded nucleic acid fragments (Maniatis, T., et al., supra., p. 146). Unless otherwise provided, ligation may be accomplished using known buffers and conditions with 10 units to T4 DNA ligase (“ligase”) per 0.5 μg of approximately equimolar amounts of the DNA fragments to be ligated.

The polypeptides and polynucleotides of the present invention are preferably provided in an isolated form, and preferably are purified to homogeneity.

A “replicon” is any genetic element (e.g., plasmid, chromosome, virus) that functions as an autonomous unit of DNA replication in vivo; i.e., capable of replication under its own control.

A “vector” is a repilcon, such as a plasmid, phage, or cosmid, to which another DNA segment may be attached so as to bring about the replication of the attached segment.

A “double-stranded DNA molecule” refers to the polymeric form of deoxyribonucleotides (bases adenine, guanine, thymine, or cytosine) in a double-stranded helix, both relaxed and supercoiled. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having the sequence homologous to the mRNA).

A DNA “coding sequence of” or a “nucleotide sequence encoding” a particular protein, is a DNA sequence which is transcribed and translated into a polypeptide when placed under the control of appropriate regulatory sequences.

A “promoter sequence” is a DNA regulatory region capable of binding RNA polymerase in a cell and initiating transcription of a downstream (3′ direction) coding sequence. For purposes of defining the present invention, the promoter sequence is bound at the 3′ terminus by a translation start codon (e.g., ATG) of a coding sequence and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain the −10 and −35 consensus sequences.

DNA “control sequences” refers collectively to promoter sequences, ribosome binding site, polyadenylation signals, transcription termination sequences, upstream regulatory domains, enhancers, and the like, which collectively provide for the expression (i.e., the transcription and translation) of a coding sequence in a host cell.

A control sequence “directs the expression” of a coding sequence in a cell when RNA polymerase will bind the promoter sequence and transcribe the coding sequence into mRNA, which is then translated into the polypeptide encoded by the coding sequence.

A “host cell” is a cell which has been transformed or transfected, or is capable of transformation or transfection by an exogenous DNA sequence.

A cell has been “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed or transfected cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cell containing the exogenous DNA.

A “clone” is a population of cells derived from a single cell or common ancestor by mitosis. A “cell line” is a clone of a primary cell that is capable of stable growth in vitro for many generations.

A “heterologous” region of a DNA construct is an identifiable segment of DNA within or attached to another DNA molecule that is not found in association with the other molecule in nature.

Further Description and Definitions:

The coding region of the RNaseP RNA structural and protein genes may be isolated, for example, by screening using the deposit containing a S. pneumoniae 0100993 strain which has been deposited with the National Collections of Industrial and Marine Bacteria Ltd. (herein “NCIMB”), 23 St. Machar Drive, Aberdeen AB2 1 RY, Scotland on Apr. 11, 1996 and assigned NCIMB Deposit No. 40794. It was referred to as S. pneumoniae 0100993 on deposit. The S. pneumoniae strain deposit is referred to herein as “the deposited strain” or as “the DNA of the deposited strain.”

The deposited strain contains the full length RNaseP catalytic RNA structural gene as well as the RNase P protein gene. The sequence of the polynucleotides contained in the deposited strain, as well as the amino acid sequence of the polypeptide encoded thereby or RNA transcribed thereby, are controlling in the event of any conflict with any description of sequences herein.

The deposit of the deposited strain has been made under the terms of the Budapest Treaty on the International Recognition of the Deposit of Micro-organisms for Purposes of Patent Procedure. The strain will be irrevocably and without restriction or condition released to the public upon the issuance of a patent. The deposited strain is provided merely as convenience to those of skill in the art and is not an admission that a deposit is required for enablement, such as that required under 35 U.S.C. §112.

A license may be required to make, use or sell the deposited strain, and compounds derived therefrom, and no such license is hereby granted.

The nucleotide sequences disclosed herein can also be obtained by synthetic chemical techniques known in the art or can be obtained from S. pneumoniae 0100993 by probing a DNA preparation with probes constructed from the particular sequences disclosed herein. Alternatively, oligonucleotides derived from a disclosed sequence can act as PCR primers in a process of PCR-based cloning of the sequence from a bacterial genomic source. It is recognized that such sequences will also have utility in diagnosis of the type of infection the pathogen has attained.

A polynucleotide of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence which encodes the polypeptide may be identical to the coding sequence shown or may be a different coding sequence which coding sequence, as a result of the redundancy or degeneracy of the genetic code, encoding the same polypeptide.

Thus, the term “polynucleotide encoding a polypeptide” encompasses a polynucleotide which includes only coding sequence for the polypeptide as well as a polynucleotide which includes additional coding and/or non-coding sequence.

The present invention therefore includes polynucleotides, wherein the coding sequence for the mature polypeptide may be fused in the same reading frame to a polynucleotide sequence which aids in expression and secretion of a polypeptide from a host cell, for example, a leader sequence which functions as a secretory sequence for controlling transport of a polypeptide from the cell. The polypeptide having a leader sequence is a preprotein and may have the leader sequence cleaved by the host cell to form the mature form of the polypeptide. The polynucleotides may also encode for a proprotein which is the mature protein plus additional 5′ amino acid residues. A mature protein having a prosequence is a proprotein and is an inactive form of the protein. Once the prosequence is cleaved an active mature protein remains.

Thus, for example, the polynucleotide of the present invention may encode for a mature protein, or for a protein having a prosequence or for a protein having both a prosequence and a presequence (leader sequence). Further, the amino acid sequences provided herein show a methionine residue at the NH₂-terminus. It is appreciated, however, that during post-translational modification of the peptide, this residue may be deleted. Accordingly, this invention contemplates the use of both the methionine-containing and the methionineless amino terminal variants of each protein disclosed herein.

The polynucleotides of the present invention may also have the coding sequence fused in frame to a marker sequence at either the 5′ or 3′ terminus of the gene which allows for purification of the polypeptide of the present invention. The marker sequence may be a hexa-histidine tag supplied by the pQE series of vectors (supplied commercially by Quiagen Inc.) to provide for purification of the polypeptide fused to the marker in the case of a bacterial host. Alternatively the maltose binding protein (MBP) fusion system may be employed. In this system the gene of interest is fused the ma1E gene encoding the MBP (supplied by New England BioLabs). The fusion product is purified in a one step procedure based on the MBP affinity for maltose. A pre-engineered Xa cleavage site allows for efficient removal of the MBP component from the gene product of interest.

### DETAILED DESCRIPTION OF THE INVENTION

The ribonucleoprotein, RNase P, plays a key role in the biosynthesis of transfer RNA (tRNA), itself a key intermediate in protein biosynthesis. RNase P functions to process precursor tRNAs into mature tRNAs by endoribonucleolytic action. The complex in the prokaryotes studied is composed of two subunits: a catalytic RNA and protein co-factor. Recent reviews of certain RNase P molecules exist. See for example, L. A. Kirsebom, Molecular Microbiology 17(3), 411-420 (1995) or N. R. Pace and J. W. Brown, J. Bacteriol. 177 (8), 1919-1928 (1995).

The invention relates to novel RNase P polynucleotides as described in greater detail below. In particular, the invention relates to polynucleotides of a novel RNaseP of S. pneumoniae, which is related by structure and nucleotide sequence homology to RNase P RNA genes set forth in FIGS. 1 and 2. The invention relates especially to RNaseP having the nucleotide sequence set out in Table 1 [SEQ ID NO:3 or 7, each of which are slight, functional variants of each other], and to the RNaseP nucleotide sequences of the DNA in the deposited strain and RNAs transcribed therefrom. The invention also relates to the RNase P RNA component, particularly in its catalytic form, and sequences from which such component is transcribed.

RNase P RNA Component:

Phylogenetic comparisons readily allow secondary structure modeling and the identification of a minimum consensus structure. Data concerning the RNase P RNA structure are available, for example, in the RNase P database on the w.w.w.(http://jwbrown.mbio.ncsu.edu/RNaseP/home.html). Polynucleotides of the invention from which the RNA component is transcribed are set forth in Table 1 [SEQ ID NO: 3 and 7].

In general, few nucleotides are conserved but compensatory base changes in hydrogen bonded regions show that the overall structure is preserved throughout eubacteria. Universal conservation of primary sequences (E. coli: 61-74, 353-360) together with other conserved or quasi-conserved nucleotides implicate functional importance, the significance of which remain unknown. To date all RNase P RNA molecules can be folded to fit a consensus ‘cage-like’ structure, beyond this domain there is no convincing structural similarity between prokaryotic and eukaryotic RNase P RNAs.

RNase P Protein Component:

The precise functional role of the protein remains unknown. While it is appreciated that in vitro the experimental conditions can be established such that the protein component is not necessary for catalytic activity, in vivo, the protein components appears to be required. The present invention provides that the RNase P protein component from S. aureus, B. subtilis, E. coli and other bacteria among other RNase P protein components, may be used with the RNaseP RNA of the invention. These components will complement the activity of this RNA when expressed in a bacteria, particularly in vivo in bacteria other than S. pneumoniae. S. pneumoniae RNaseP protein components may also be used with the RNaseP RNA of the invention (see SEQ ID NO:5 and the coding sequence of SEQ ID NO:6). RNaseP protein from Staphylococcus areas, such as one having the sequence set forth in SEQ ID NO:2, may also be used in combination with a RNaseP RNA from Streptococcus pneumoniae, such as the sequence of SEQ ID NO:3 or 7. RNaseP protein of other bacteria may also be used in this manner.

The full length sequence encoding the intact RNase P protein component can be obtained by probing a genomic library using degenerate primers made from the S. aureus or S. pneumoniae RNaseP amino acid sequence and polynucleotide sequences set forth below in Table 1.

TABLE 1
RNase P Polynucleotide and Polypeptide Sequences

(A) Sequences from S. aureus RNaseP polynucleotide
sequence [SEQ ID NO:1].
5′-ATG TTA TTG GAA AAA GCT TAC CGA ATT AAA AAG AAT GCA
GAT TTT CAG AGA ATA TAT AAA AAA GGT CAT TCT GTA GCC AAC
AGA CAA TTT GTT GTA TAC ACT TGT AAT AAT AAA GAA ATA GAC
CAT TTT CGC TTA GGT ATT AGT GTT TCT AAA AAA CTA GGT AAT
GCA GTG TTA AGA AAC AAG ATT AAA AGA GCA ATA CGT GAA AAT
TTC AAA GTA CAT AAG TCG CAT ATA TTG GCC AAA GAT ATT ATT
GTA ATA GCA AGA CAG CCA GCT AkA GAT ATG ACG ACT TTA CAA
ATA CAG AAT AGT CTT GAG CAC GTA CTT AAA ATT GCC AAA CTT
TTT AAT AAA AAG ATT AAG TAA- 3′
(B) S. aureus RNaseP polypeptide sequence deduced
from the polynucleotide sequence in this table
[SEQ ID NO:2].
NH₂- MLLEK VYRTK KNADE GRIYK KGHSV ANRQF VVYTC NNKET
DHFRL GTSVS KKLGN AVLRN KTKRA TRENF KVHKS HILAK DITVI
ARQPA KDMTT LQTQN SLEHV LKIAK VFNKK IK-COOH
(C) Polynucleotide sequence embodiments [SEQ ID NO:3].
X-(R₁)ₙ₋
GATGTGCAATTTTTGGATAATCGAGTGAAGGGAATTGCTTCTCATGAGGAAAGTCC
ATGATAGCACAGGCTGTGATGCCTGTAGTATTTGTGATAGGCGAAACCATAAGCCT
AGGGACGAGAATTCGTTACGGCAGTTGAAATGGATAAGTCCTTGGATAAGCCAGAG
TAGGCTTGAAAGTGCCACAGTGACGGAGTCTTTCTGGAAACAGAGAGAGTGGAACG
CGGTAAACCCCTCAAGCTAGCAACCCAAATTTTGGTCGGGGCATGGAGTACGCGGA
AACGGACGTAGTATTCTGACTGGTATCAGCTAGAGCTGTTAGTGGTAGACAGATGA
TTATCGAAGGAAGTGGTCCTAGTCACTTTTGGAACAAAACATGGCTTATAGAAAAT
TGCATATAGG-(R₂)ₙ-Y
(D) RNaseP RNA gene sequence [SEQ ID NO:3].
5′-
GATGTGCAATTTTTGGATAATCGAGTGAAGGGAATTGCTTCTCATGAGGAAAGTCC
ATGATAGCACAGGCTGTGATGCCTGTAGTATTTGTGATAGGCGAAACCATAAGCCT
AGGGACGAGAATTCGTTACGGCAGTTGAAATGGATAAGTCCTTGGATAAGCCAGAG
TAGGCTTGAAAGTGCCACAGTGACGGAGTCTTTCTGGAAACAGAGAGAGTGGAACG
CGGTAAACCCCTCAAGCTAGCAACCCAAATTTTGGTCGGGGCATGGAGTACGCGGA
AACGGACGTAGTATTCTGACTGGTATCAGCTAGAGCTGTTAGTGGTAGACAGATGA
TTATCGAAGGAAGTGGTCCTAGTCACTTTTGGAACAAAACATGGCTTATAGAAAAT
TGCATATAGG-3′
(E) RNaseP RNA gene (5′-halF) [SEQ ID NO:4].
5′-
GATGTGCAATTTTTGGATAATCGCGTGAGGAGAATTGCTTCTCATGAGGAAAGTCC
ATGCTAGCACAGGCTGTGATGCCTGTAGTGTCTGTGCTACGCGAAACCATAAGCCT
ATGGACGAGAAATCGTTACCGCAGTTGAAATGGCTAATTCCTTGGATAAGCCACAG
TATGCTTGAAATTGCCACAGTGACCGAGTCTTTCTGGAAACACATAGATTGGAACG
CCGTAACCCCTCAAGCTAGCAACCCAAAATTTTGGTCGGGGCA-3′
(F) RNaseP RNA gene (5′-half) [SEQ ID NO:4]
sequence embodiment.
X-(R₁)ₙ₋
GATGTGCAATTTTTGGATAATCGCGTGAGGAGAATTGCTTCTCATGAGGAAAGTCC
ATGCTAGCACAGGCTGTGATGCCTGTAGTGTCTGTGCTACGCGAAACCATAAGCCT
ATGGACGAGAAATCGTTACGGCAGTTGAAATGGCTAATTCCTTGGATAAGCCACAG
TATGCTTGAAATTGCCACAGTGACCGAGTCTTTCTGGAAACACATAGATTGGAACG
CCGTAACCCCTCAAGCTAGCAACCCAAATTTTGGTCGGGGCA-(R₂)ₙ-Y
(G) RNaseP Protein Coding Sequence [SEQ ID NO:5].
5′-
TTGAAGAAAAACTTTCGTGTAAAAAGAGAGAAAGATTTTAAGGCGATTTTCAAGGA
GGGGACAAGTTTTGCTAATCGCAAATTTGTGGTCTACCAATTAGAAAACCAGAAAA
ACCATTTTCGAGTAGGTCTATCAGTTAGCAAAAAACTGGGGAATGCCGTCACTAGA
AATCAAATTAAGCGACGGATTCGGCATATTATCCAGAATGCAAAAGGGAGTCTGGT
AGAAGATGTCGACTTTGTTGTCATTGCTCGAAAAGGAGTCGAAACCTTGGGATACG
CAGAGATGGAGAAAAATCTACTCCATGTATTAAAATTATCAAAGATTTACCGGGAA
-3′
(H) RNaseP Protein [SEQ ID NO:6]
NH₂-
LKKNFRVKREKDFKAIFKEGTSFANRKFVVYQLENQKNHFRVGLSVSKKLGNAVTR
NQIKRRIRHIIQNAKGSLVEDVDFVVIARKGVETLGYAEMEKNLLHVLKLSKIYRE
-COOH
(I) RNaseP Protein Coding Sequence [SEQ ID NO:5].
X-(R₁)ₙ₋
TTGAAGAAAAACTTTCGTGTAAAAAGAGAGAAAGATTTTAAGGCGATTTTCAAGGA
GGGGACAAGTTTTGCTAATCGCAAATTTGTGGTCTACCAATTAGAAAACCAGAAAA
ACCATTTTCGAGTAGGTCTATCAGTTAGCAAAAAACTGGGGAATGCCGTCACTAGA
AATCAAATTAAGCGACGGATTCGGCATATTATCCAGAATGCAAAAGGGAGTCTGGT
AGAAGATGTCGACTTTGTTGTCATTGCTCGAAAAGGAGTCGAAACCTTGGGATACG
CAGAGATGGAGAAAAATCTACTCCATGTATTAAAATTATCAAAGATTTACCGGGAA-
(R₂)ₙ-Y
(J) RNaseP Protein [SEQ ID NO:6]
X-(R₁)ₙ-
LKKNFRVKREKDFKAIFKEGTSFANRKFVVYQLENQKNHFRVGLSVSKKLGNAVTR
NQIKRRIRHIIQNAKGSLVEDVDFVVIARKGVETLGYAEMEKNLLHVLKLSKIYRE-
(R₂)ₙ-Y
(K) Full Length RNaseP RNA gene [SEQ ID NO:7].
5′-GAT GTG CAA TTT TTG GAT AAT CGC GTG AGG AGA ATT GCT
TCT CAT GAG GAA AGT CCA TGC TAG CAC AGG CTC TGA TGC CTG
TAG TGT TTG TGC TAG GCG AAA CCA TAA GCC TAG GGA CGA GAA
ATC GTT ACG GCA GTT GAA ATG GCT AAG TCC TTG GAT AAG CCA
GAG TAG GCT TGA AAG TGC CAC AGT GAC GGA GTC TTT CTC CAA
ACA GAG AGA GTG CAA CGC GCT AAA CCC CTC AAC CTA GCA ACC
CAA ATT TTG GTC GGG GCA TGG AGT ACG CGG AAA CGA ACG TAG
TAT TCT GAC TGC TAT CAG CTA GAC CTG TTA GTG GTA GAC AGA
TGA TTA TCG AAG GAA GTG GTC CTA GTC ACT TCT GGA ACA AAA
CAT GGC TTA TAG AAA ATT GCA TAT AGG-3′
(L) RNaseP RNA gene [SEQ ID NO:7] sequence embodiment.
X-(R₁)ₙ-GAT GTG CAA TTT TTG GAT AAT CGC GTG AGG AGA ATT
GCT TCT CAT GAG GAA AGT CCA TGC TAG CAC AGG CTG TGA TGC
CTG TAG TGT TTG TGC TAG GCG AAA CCA TAA GCC TAG GGA CGA
GAA ATC GTT ACG GCA GTT GAA ATG GCT AAG TCC TTG GAT AAG
CCA GAG TAG GCT TGA AAG TGC CAC AGT GAC GGA GTC TTT CTG
GAA ACA GAG AGA GTG GAA CGC GGT AAA CCC CTC AAG CTA GCA
ACC CAA ATT TTG GTC GGG GCA TGG AGT ACG CGG AAA CGA ACG
TAG TAT TCT GAC TGC TAT CAG CTA GAG CTG TTA GTG GTA GAC
AGA TGA TTA TCG AAG GAA GTG GTC CTA GTC ACT TCT GGA ACA
AAA CAT GGC TTA TAG AAA ATT GCA TAT AGG-(R₂)ₙ-Y

Polypeptides of the invention:

The polypeptides of the invention include the polypeptide isolated from S. pneumoniae, particularly the deposited strain, such as the sequences set forth in Table 1 [SEQ ID NO:6], and also sequences isolated using, for example, the S. aureus sequences set forth in of Table 1 [SEQ ID NOS:1 and 2]. In particular the mature polypeptide as well as polypeptides and fragments, particularly those which have the biological activity of RNaseP, and also those which have at least 50%, 60% or 70% identity to the polypeptide of Table 1 [SEQ ID NO:2] or the relevant portion, preferably at least 80% identity to the polypeptide of Table 1 [SEQ ID NO:2], and more preferably at least 90% similarity (more preferably at least 90% identity) to the polypeptide of Table 1 [SEQ ID NO:2 or 6] and still more preferably at least 95% similarity (still more preferably at least 95% identity) to the polypeptide of Table I [SEQ ID NO:2 or 6] and also include portions of such polypeptides with such portion of the polypeptide generally containing at least 30 amino acids and more preferably at least 50 amino acids.

A fragment is a variant polypeptide having an amino acid sequence that entirely is the same as part but not all of the amino acid sequence of the aforementioned polypeptides. As with RNaseP polypeptides fragments may be “free-standing,” or comprised within a larger polypeptide of which they form a part or region, most preferably as a single continuous region, a single larger polypeptide.

Preferred fragments include, for example, truncation polypeptides having a portion of an RNaseP amino acid sequence of SEQ ID NO:6. Also included are those sequences obtained using or derived from a sequence of Table 1 [SEQ ID NOS: 1, 2, 5 or 6], or of variants thereof, such as a continuous series of residues that includes the amino terminus, or a continuous series of residues that includes the carboxyl terminus. Degradation forms of the polypeptides of the invention in a host cell or bacteria, particularly a S. pneumoniae, are also preferred. Further preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions.

Also preferred are biologically active fragments which are those fragments that mediate activities of RNaseP, including those with a similar activity or an improved activity, or with a decreased undesirable activity. Also included are those fragments that are antigenic or immunogenic in an animal, especially in a human. Particularly preferred are fragments comprising receptors or domains of enzymes that confer a function essential for viability of S. pneumoniae or the ability to initiate, or maintain cause disease in an individual, particularly a human.

Variants that are fragments of the polypeptides of the invention may be employed for producing the corresponding full-length polypeptide by peptide synthesis; therefore, these variants may be employed as intermediates for producing the full-length polypeptides of the invention.

Polypeptides of the invention may also be used to assess the binding of small molecule substrates and ligands in, for example, cells, cell-free preparations, chemical libraries, and natural product mixtures. These substrates and ligands may be natural substrates and ligands or may be structural or functional mimetics. See, e.g., Coligan et al., Current Protocols in Immunology 1(2): Chapter 5 (1991).

Polynucleotides of the invention:

Another aspect of the invention relates to isolated polynucleotides, including the full length gene, that encode the RNaseP polypeptide of the invention [SEQ ID NO:6], such as those isolated using a sequence of Table 1 [SEQ ID NOS: 1, 2, 5 or 6], such as by amplification using degenerate primers made from these sequences, and polynucleotides closely related thereto and variants thereof.

Using the information provided herein, such as a polynucleotide sequence derived from a sequence set out in SEQ ID NOS: 1, 2, 3, 4, 5 or 7, a polynucleotide of the invention encoding RNaseP polypeptide or RNA (such as that transcribed from SEQ ID NO:3 or 7) may be obtained using standard cloning and screening methods, such as those for cloning and sequencing chromosomal DNA fragments from bacteria using S. pneumoniae 0100993 cells as starting material, followed by obtaining a full length clone. For example, to obtain a polynucleotide sequence of the invention, such as a sequence given in SEQ ID NO:3 or 7 or an RNaseP structural protein gene, typically a library of clones of chromosomal DNA of S. pneumoniae 0100993 in E.coli or some other suitable host or bacteria is probed with a radiolabeled oligonucleotide, preferably a polymer or longer, derived from a partial sequence, derived, for example, from a polynucleotide sequence of SEQ ID NO: 1, 3, 4, 5 or 7, as appropriate. Clones carrying DNA identical to that of the probe can then be distinguished using stringent conditions. By sequencing the individual clones thus identified with sequencing primers designed from the original sequence it is then possible to extend the sequence in both directions to determine the full gene sequence. Conveniently, such sequencing is performed using denatured double stranded DNA prepared from a plasmid clone. Suitable techniques are described by Maniatis, T., Fritsch, E. F. and Sambrook et al., MOLECULAR CLONING, A LABORATORY MANUAL, 2nd Ed.; Cold Spring Harbor Laboratory Press, Cold Spring Harbor, N.Y. (1989). (see in particular Screening By Hybridization 1.90 and Sequencing Denatured Double-Stranded DNA Templates 13.70). Illustrative of the invention, the polynucleotides set out in SEQ ID NO:3 and 7 were discovered in a DNA library derived from S. pneumoniae 0100993.

The invention provides the coding sequence for the mature polypeptide or a fragment thereof, by itself as well as the coding sequence for the mature polypeptide or a fragment in reading frame with other coding sequence, such as those encoding a leader or secretory sequence, a pre-, or pro- or prepro-protein sequence. The polynucleotide may also contain non-coding sequences, including for example, but not limited to non-coding 5′ and 3′ sequences, such as the transcribed, non-translated sequences, termination signals, ribosome binding sites, sequences that stabilize mRNA, introns, polyadenylation signals, and additional coding sequence which encode additional amino acids. For example, a marker sequence that facilitates purification of the fused polypeptide can be encoded. In certain embodiments of the invention, the marker sequence is a hexa-histidine peptide, as provided in the pQE vector (Qiagen, Inc.) and described in Gentz et al., Proc. Natl. Acad. Sci., USA 86: 821-824 (1989), or an HA tag (Wilson et al., Cell 37: 767 (1984). Polynucleotides of the invention also include, but are not limited to, polynucleotides comprising a structural gene and its naturally associated sequences that control gene expression.

The invention also includes polynucleotides of the formula set forth in Table I (C)[SEQ ID NO:3] and (K)[SEQ ID NO:7] wherein, at the 5′ end of the molecule, X is hydrogen, and at the 3′ end of the molecule, Y is hydrogen or a metal, R₁ and R₂ is any nucleic acid residue, and n is an integer between 1 and 3000. Any stretch of nucleic acid residues denoted by either R group, where R is greater than 1, may be either a heteropolymer or a homopolymer, preferably a heteropolymer. A preferred embodiment for the sequence set forth in Table 1 (C) [SEQ ID NO:3] or (K)[SEQ ID NO:7] has R₁ or R₂ being between 1 and 10 or 1 and 20, and especially being 1, 2, 3, 4, 5, 6, 7, 8, 9 or 10. The invention also provides RNA transcribed from such polynucleotides, particularly catalytic RNAs.

The term “polynucleotide encoding a polypeptide” as used herein encompasses polynucleotides that include a sequence encoding a polypeptide of the invention, particularly a bacterial polypeptide and more particularly a polypeptide of the S. pneumoniae RNaseP [such as SEQ ID NO:6], particularly those obtained using an amino acid or polynucleotide sequence set out in Table 1 [SEQ ID NOS: 1-7]. The term also encompasses polynucleotides that include a single continuous region or discontinuous regions encoding the polypeptide (for example, interrupted by integrated phage or an insertion sequence or editing) together with additional regions, that also may contain coding and/or non-coding sequences.

The invention further relates to variants of the polynucleotides described herein that encode for variants of the polypeptide of the invention. Variants that are fragments of the polynucleotides of the invention may be used to synthesize full-length polynucleotides of the invention.

Further particularly preferred embodiments are polynucleotides encoding RNaseP variants, that have the amino acid sequence of RNaseP polypeptide in which several, a few, 5 to 10, 1 to 5, 1 to 3, 2, 1 or no amino acid residues are substituted, deleted or added, in any combination. Especially preferred among these are silent substitutions, additions and deletions, that do not alter the properties and activities of RNaseP.

Further preferred embodiments of the invention are polynucleotides that are at least 50%, 60% or 70% identical over their entire length to a polynucleotide encoding RNaseP polypeptide [SEQ ID NO:6], particularly those obtained using the amino acid or polynucleotide sequence set out in Table 1 [SEQ ID NOS: 1-7], and polynucleotides that are complementary to such polynucleotides. Alternatively, most highly preferred are polynucleotides that comprise a region that is at least 80% identical over its entire length to a polynucleotide encoding RNaseP polypeptide of the deposited strain and polynucleotides complementary thereto. In this regard, polynucleotides at least 90% identical over their entire length to the same are particularly preferred, and among these particularly preferred polynucleotides, those with at least 95% are especially preferred. Furthermore, those with at least 97% are highly preferred among those with at least 95%, and among these those with at least 98% and at least 99% are particularly highly preferred, with at least 99% being the more preferred.

Preferred embodiments of the invention are polynucleotides that are at least 50%, 60% or 70% identical over their entire length to an RNaseP polynucleotide having a nucleotide sequence set out in SEQ ID NOS:3, 4 or 7 and polynucleotides that are complementary to such polynucleotides. Alternatively, most highly preferred are polynucleotides that comprise a region that is at least 80% identical over its entire length to an RNaseP polynucleotide of the deposited strain and polynucleotides complementary thereto. In this regard, polynucleotides at least 90% identical over their entire length to the same are particularly preferred, and among these particularly preferred polynucleotides, those with at least 95% are especially preferred. Furthermore, those with at least 97% are highly preferred among those with at least 95%, and among these those with at least 98% and at least 99% are particularly highly preferred, with at least 99% being the more preferred. It is especially preferred that these polynucleotides be RNAs, especially catalytic RNAs.

Preferred embodiments are polynucleotides that encode polypeptides that retain substantially the same biological function or activity as the mature polypeptide encoded by the DNA of the invention or as the RNase P RNA component transcribed by the DNA of SEQ ID NO:3, 4or 7.

The invention further relates to polynucleotides that hybridize to the herein above-described sequences. In this regard, the invention especially relates to polynucleotides that hybridize under stringent conditions to the herein above-described polynucleotides. As herein used, the terms “stringent conditions” and “stringent hybridization conditions” mean hybridization will occur only if there is at least 95% and preferably at least 97% identity between the sequences. An example of stringent hybridization conditions is overnight incubation at 42° C. in a solution comprising: 50% formamide, 5×SSC (150 mM NaCl, 15 mM trisodium citrate), 50 mM sodium phosphate (pH7.6), 5×Denhardt's solution, 10% dextran sulfate, and 20 micrograms/ml denatured, sheared salmon sperm DNA, followed by washing the hybridization support in 0.1×SSC at about 65° C. Hybridization and wash conditions are well known and exemplified in Sambrook, et al.. Molecular Cloning: A Laboratory Manual, Second Edition, Cold Spring Harbor, N.Y., (1989), particularly Chapter 11 therein.

The invention also provides a polynucleotide consisting essentially of a polynucleotide sequence obtainable by screening an appropriate library containing the complete gene for a polynucleotide sequence of the invention using, as a probe, a polynucleotide set forth in Table 1, or fragment thereof, under stringent hybridization conditions; and isolating said DNA sequence. Fragments useful for obtaining such a polynucleotide include, for example, probes and primers described elsewhere herein.

As discussed herein regarding polynucleotide assays of the invention, for instance, polynucleotides of the invention as discussed above, may be used as a hybridization probe for RNA, cDNA and genomic DNA to isolate full-length cDNAs and genomic clones encoding RNaseP and to isolate cDNA and genomic clones of other genes that have a high sequence similarity to the RNaseP gene. Such probes generally will comprise at least 15 bases. Preferably, such probes will have at least 30 bases and may have at least 50 bases. Particularly preferred probes will have at least 30 bases and will have 50 bases or less.

Polynucleotides of the invention that are oligonucleotides derived from the sequences of SEQ ID NOS:1 and/or 2 and/or 3 and/or 4 and/or 5 and/or 6 and/or 7 may be used in the processes herein as described, but preferably for PCR, to determine whether or not the polynucleotides identified herein in whole or in part are transcribed in bacteria in infected tissue. It is recognized that such sequences will also have utility in diagnosis of the stage of infection and type of infection the pathogen has attained.

The invention also provides polynucleotides that may encode a polypeptide that is the mature protein plus additional amino or carboxyl-terminal amino acids, or amino acids interior to the mature polypeptide (when the mature form has more than one polypeptide chain, for instance). Such sequences may play a role in processing of a protein from precursor to a mature form, may allow protein transport, may lengthen or shorten protein half-life or may facilitate manipulation of a protein for assay or production, among other things. As generally is the case in vivo, the additional amino acids may be processed away from the mature protein by cellular enzymes.

A precursor protein, having the mature form of the polypeptide fused to one or more prosequences may be an inactive form of the polypeptide. When prosequences are removed such inactive precursors generally are activated. Some or all of the prosequences may be removed before activation. Generally, such precursors are called proproteins.

In sum, a polynucleotide of the invention may encode a mature protein, a mature protein plus a leader sequence (which may be referred to as a preprotein), a precursor of a mature protein having one or more prosequences that are not the leader sequences of a preprotein, or a preproprotein, which is a precursor to a proprotein, having a leader sequence and one or more prosequences, which generally are removed during processing steps that produce active and mature forms of the polypeptide.

Cloning of S. pneumoniae RNase P RNA structural gene:

A homolog to B. subtilis RNase P RNA was identified in a proprietary S. pneumoniae database comprising sequences from randomly sequenced S. pneumoniae DNA library in an E. coli host. This homolog is shown in Table 1 [SEQ ID NO:3 and 7, each being an embodiment of the homolog].

Preparation of the RNase P protein component:

The present invention also relates to vectors which include polynucleotides of the present invention, host cells which are genetically engineered with vectors of the invention and the production of polypeptides of the invention by recombinant techniques.

In accordance with yet a further aspect of the present invention, there is therefore provided a process for producing the polypeptide of the invention by recombinant techniques by expressing a polynucleotide encoding said polypeptide in a host and recovering the expressed product. Alternatively, the polypeptides of the invention can be synthetically produced by conventional peptide synthesizers.

Host cells are genetically engineered (transduced or transformed or transfected) with the vectors of this invention which may be, for example, a cloning vector or an expression vector. The vector may be, for example, in the form of a plasmid, a cosmid, a phage, etc. The engineered host cells can be cultured in conventional nutrient media modified as appropriate for activating promoters, selecting transformants or amplifying the genes. The culture conditions, such as temperature, pH and the like, are those previously used with the host cell selected for expression, and will be apparent to the ordinarily skilled artisan.

Suitable expression vectors include chromosomal, nonchromosomal and synthetic DNA sequences, e.g., bacterial plasmids; phage DNA; baculovirus; yeast plasmids; vectors derived from combinations of plasmids and phage DNA. However, any other vector may be used as long as it is replicable and viable in the host.

For recombinant production, host cells can be genetically engineered to incorporate expression systems or portions thereof or polynucleotides of the invention. Introduction of a polynucleotide into the host cell can be effected by methods described in many standard laboratory manuals, such as Davis et al., BASIC METHODS IN MOLECULAR BIOLOGY, (1986) and Sambrook et al., MOLECULAR CLONING: A LABORATORY MANUAL, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, transduction, scrape loading, ballistic introduction and infection.

Representative examples of appropriate hosts include bacterial cells, such as streptococci, staphylococci, enterococci E. coli, streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells.

A great variety of expression systems can be used to produce the polypeptides of the invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., MOLECULAR CLONING, A LABORATORY MANUAL, (supra).

The appropriate DNA sequence may be inserted into the vector by a variety of procedures. In general, the DNA sequence is inserted into an appropriate restriction endonuclease site(s) by procedures known in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli. lac or trp, the phage lambda P_{L} promoter and other promoters known to control expression of genes in eukaryotic or prokaryotic cells or their viruses. The expression vector may also contains a ribosome binding site for translation initiation and/or a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The gene can be placed under the control of a promoter, ribosome binding site (for bacterial expression) and, optionally, an operator (collectively referred to herein as “control” elements), so that the DNA sequence encoding the desired protein is transcribed into RNA in the host cell transformed by a vector containing this expression construction. The coding sequence may or may not contain a signal peptide or leader sequence. The polypeptides of the present invention can be expressed using, for example, the E. Coli tac promoter or the protein A gene (spa) promoter and signal sequence. Leader sequences can be removed by the bacterial host in post-translational processing. See, e.g., U.S. Pat. Nos. 4,431,739; 4,425,437; 4,338,397. Promoter regions can be selected from any desired gene using CAT (chloramphenicol transferase) vectors or other vectors with selectable markers. Two appropriate vectors are PKK232-8 and PCM7. Particular named bacterial promoters include lac, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In addition to control sequences, it may be desirable to add regulatory sequences which allow for regulation of the expression of the protein sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the coding sequences may be desirable to achieve this end. For example, in some cases it may be necessary to modify the sequence so that it may be attached to the control sequences with the appropriate orientation; i.e., to maintain the reading frame. The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

Generally, recombinant expression vectors will include origins of replication and selectable markers permitting transformation of the host cell. e.g., the ampicillin resistance gene of E. coli and S. cerevisiae TRPI gene, and a promoter derived from a highly-expressed gene to direct transcription of a downstream structural sequence. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

The vector containing the appropriate DNA sequence as hereinabove described, as well as an appropriate promoter or control sequence, may be employed to transform an appropriate host to permit the host to express the protein.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example. Bacterial: pET-3 vectors (Stratagene), pQE70, pQE60, pQE-9 (Qiagen), pbs, pD10, phagescript, psiX174, pbluescript SK, pbsks, pNH8A, pNH16a, pNH18A, pNH46A (Stratagene); ptrc99a, pKK223-3, pKK233-3, pDR540, pRIT5 (Pharmacia). Eukaryotic: pBlueBacIII (Invitrogen), pWLNEO, pSV2CAT, pOG44, pXT1, pSG (Stratagene) pSVK3, pBPV, pMSG, pSVL (Pharmacia). However, any other plasmid or vector may be used as long as they are replicable and viable in the host.

Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), a baculovirus insect cell system, YCp19 (Saccharomyces). See. generally, “DNA Cloning”: Vols. I & II. Glover et al. ed. IRL Press Oxford (1985) (1987) and; T. Maniatis et al. (“Molecular Cloning” Cold Spring Harbor Laboratory (1982).

In some cases, it may be desirable to add sequences which cause the secretion of the polypeptide from the host organism, with subsequent cleavage of the secretory signal.

Polypeptides can be expressed in host cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook. et al.. Molecular Cloning: A Laboratory Manual, Second Edition, Cold Spring Harbor, N.Y., (1989), the disclosure of which is hereby incorporated by reference.

Following transformation of a suitable host strain and growth of the host strain to an appropriate cell density, the selected promoter is induced by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well known to those skilled in the art.

Depending on the expression system and host selected, the polypeptide of the present invention may be produced by growing host cells transformed by an expression vector described above under conditions whereby the polypeptide of interest is expressed. The polypeptide is then isolated from the host cells and purified. If the expression system secretes the polypeptide into growth media, the polypeptide can be purified directly from the media. If the polypeptide is not secreted, it is isolated from cell lysates or recovered from the cell membrane fraction. Where the polypeptide is localized to the cell surface, whole cells or isolated membranes can be used as an assayable source of the desired gene product. Polypeptide expressed in bacterial hosts such as E. coli may require isolation from inclusion bodies and refolding. Where the mature protein has a very hydrophobic region which leads to an insoluble product of overexpression, it may be desirable to express a truncated protein in which the hydrophobic region has been deleted. The selection of the appropriate growth conditions and recovery methods are within the skill of the art.

The polypeptide can be recovered and purified from recombinant cell cultures by methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

Depending upon the host employed in a recombinant production procedure, the polypeptides of the present invention may be glycosylated or may be non-glycosylated. Polypeptides of the invention may also include an initial methionine amino acid residue.

Preparation of the RNase P RNA Component:

The RNase P RNA molecules may be and have been prepared by run-off in vitro transcription using T7 RNA polymerase as according to standard conditions—usually as recommended by the supplier, e.g., Promega. The plasmid is linearized with an appropriate restriction enzyme generating a linear dsDNA comprising the full length gene encoding the RNase P RNA. The RNA is purified either from a preparative denaturing acrylamide gel or is precipitated prior to use in in vitro cleavage assays. The substrates for the RNase P RNA and the RNA complexed with its protein (RNase P protein) can be obtained by in vitro transcription of cloned genes. Useful substrates included but are not limited to pre-tRNA^{Met} or E. coli pre-4.5S or B. subtilis pre-sc RNA molecules and may be expressed using an in vitro transcription system directed by T7 RNA polymerase as described above.

Antagonists and agonists—assays and molecules

This invention provides a method of screening drugs to identify those which interfere with the RNA portion, the protein portion and/or the intact RNA/protein complex of the RNase P described herein, which method comprises measuring the interference of the activity of the protein and/or RNA by a test drug. For example since the RNA portion selected has a catalytic activity, after suitable purification and formulation the activity of the RNA can be followed by its ability to convert its natural or synthetic RNA substrates. By incorporating different chemically synthesized test compounds or natural products into such an assay of enzymatic activity one is able to detect those additives which compete with the natural or synthetic substrate or otherwise inhibit enzymatic activity.

The invention also provides a method of screening compounds to identify those which enhance (agonist) or block (antagonist) the action of RNaseP polypeptides or polynucleotides, particularly those compounds that are bacteriostatic and/or bacteriocidal. The method of screening may involve high-throughput techniques. For example, to screen for agonists or antagonists, a synthetic reaction mix, a cellular compartment, such as a membrane, cell envelope or cell wall, or a preparation of any thereof, comprising RNaseP polypeptide or polynucleotides and a labeled substrate or ligand of such polypeptide is incubated in the absence or the presence of a candidate molecule that may be a RNaseP agonist or antagonist. The ability of the candidate molecule to agonize or antagonize the RNaseP polypeptide and/or polynucleotide is reflected in decreased binding of the labeled ligand or decreased production of product from such substrate. Molecules that bind gratuitously, i.e., without inducing the effects of RNaseP polypeptide or polynucleotide are believed to be good antagonists. Molecules that bind well and increase the rate of product production from substrate are agonists. Detection of the rate or level of production of product from substrate may be enhanced by using a reporter system. Reporter systems that may be useful in this regard include but are not limited to colorimetric, fluorescent, or radiolabeled substrate converted into product, a reporter gene that is responsive to changes in RNaseP polynucleotide or polypeptide activity.

Another example of an assay for RNaseP antagonists is a competitive assay that combines RNaseP and a potential antagonist with RNaseP-binding molecules, recombinant RNaseP binding molecules, natural substrates or ligands, or substrate or ligand mimetics, under appropriate conditions for a competitive inhibition assay. RNaseP can be labeled, such as by radioactivity or a colorimetric compound, such that the number of RNaseP molecules bound to a binding molecule or converted to product can be determined accurately to assess the effectiveness of the potential antagonist.

Potential antagonists include small organic molecules, peptides, polypeptides and antibodies that bind to a polynucleotide or polypeptide of the invention and thereby inhibit or extinguish its activity. Potential antagonists also may be small organic molecules, a peptide, a polypeptide such as a closely related protein or antibody that binds the same sites on a binding molecule, such as a binding molecule, without inducing RNaseP-induced activities, thereby preventing the action of RNaseP by excluding RNaseP from binding.

Potential antagonists include a small molecule that binds to and occupies the binding site of the polypeptide and/or polynucleotide, particularly the BNase P RNA, thereby preventing binding to cellular binding molecules, such that normal biological activity is prevented. Examples of small molecules include but are not limited to small organic molecules, peptides or peptide-like molecules. Other potential antagonists include antisense molecules (see Okano, J. Neurochem. 56: 560 (1991); OLIGODEOXYNUCLEOTIDES AS ANTISENSE INHIBITORS OF GENE EXPRESSION, CRC Press, Boca Raton, Fla. (1988), for a description of these molecules). Preferred potential antagonists include compounds related to and variants of RNaseP.

Each of the DNA sequences provided herein may be used in the discovery and development of antibacterial compounds. The encoded protein, upon expression, can be used as a target for the screening of antibacterial drugs. Additionally, the DNA sequences encoding the amino terminal regions of the encoded protein or Shine-Delgarno or other translation facilitating sequences of the respective mRNA can be used to construct antisense sequences to control the expression of the coding sequence of interest.

The antagonists and agonists of the invention may be employed, for instance, to inhibit and treat disease, such as, infections of the upper respiratory tract (e.g., otitis media, bacterial tracheitis, acute epiglottitis, thyroiditis), lower respiratory (e.g., empyema, lung abscess), cardiac (e.g., infective endocarditis), gastrointestinal (e.g., secretory diarrhoea, splenic absces, retroperitoneal abscess), CNS (e.g., cerebral abscess), eye (e.g., blepharitis, conjunctivitis, keratitis, endophthaimitis, preseptal and orbital cellulitis, darcryocystitis), kidney and urinary tract (e.g., epididymitis, intrarenal and perinephric absces, toxic shock syndrome), skin (e.g., impetigo, folliculitis, cutaneous abscesses, cellulitis, wound infection, bacterial myositis) bone and joint (e.g., septic arthritis, osteomyelitis).

HTP Screening Strategies:

Assays can be developed to detect compounds that inhibit RNase P directed cleavage of RNA substrates. Several possible assay formats are suitable for HTP screening based upon the ability to incorporate labels within the RNA in a site-specific fashion by chemical synthesis. The conventional radioactive-based format is preferred, while a homogeneous fluorescence-based format is useful for subsequent follow-up of lead compounds. The use of both formats is contemplated by this invention.

Functional RNase P Assay

Biotin is introduced in to an appropriate position within the RNA substrate and the 5′ terminus labeled with ³²p. The substrate is linked via streptavidin within a 96-well plate. Following RNase P dependent hydrolysis of the substrate, the radiolabelled 5′ leader cleavage product is released into the bulk solution phase, and subject to scintillation counting. Alternatively, the RNA substrate is bound to a streptavidin-coated flashplate such that the release of the radioactive polymer into the solution phase results in a decrease in signal. This has the advantage that it is a homogeneous, continuous assay format and requires no additional manipulations after starting the assay, except for a filtration step in certain embodiments. Both formats are useful in the practice of this invention because they use the same RNA substrate.

RNA Fragment Library Rescue

An effective approach for identifying compounds that interact with RNA is contemplated. The concept is based on the over-expression of a drug binding site that is recreated on an RNA fragment, which will sequester the drug and permit the continued functioning of the intact ribozyme. This approach has recently been described in the context of a search for ligands that bind ribosomal RNA (Howard. B -A, et al., Biochem. Cell Bio. 73(11/12): 1161-1166 (1995)). Following selection the random RNA fragments that apparently present a minimal target structure for drug recognition, are incorporated into a protocol for rational drug design. Accordingly, random fragment libraries based on RNaseP RNA will be generated and used in HTP screening to identify compounds that disrupt RNA/protein interaction.

Cyclic peptide phase libraries:

The incorporation of conformational constraints into flexible lead compounds is a powerful strategy to increase lead potency and is particularly useful in the field of peptidomimetic design.(Al-Obeidi, F. et al., J. Med. Chem. 32: 2555-2561(1989);Barker, P. L., et al., J. Med. Chem. 35: 2040-2048 (1992)). Cyclization has been shown to increase the propensity for beta-turn formation in peptides the potential of which has been demonstrated by the identification of high-affinity ligands for streptavidin (Lee, M. S., et al., FEBS Lett. 359: 113-118 (1995)).

In this case, cyclic peptide libraries were constructed with flanking cysteine residues to allow efficient disulfide bond formation and cyclization during phage assembly. The streptavidin bound crystal structures of two disulfide bridged cyclic peptides showed both peptides to be in beta-turn conformations (Kahn, M. (Guest, Ed., 1993) Tetrahedron 49, Symp. 50, 3433-3677).

Beta-turns are key recognition elements in many biological interactions therefore effort has been focused on the design of small constrained beta-turn mimics (Kahn, M. (Guest Ed., 1993) Tetrahedron 49, Symp. 50, 3433-3677). This approach, when applied to RNase P, could identify cyclic peptides suitable for peptide mimic synthesis as inhibitor molecules. A cyclic octapeptide phage display library may be constructed and used to identify peptides that interact with defined RNA domains.

Secondary Evaluation

SELEX: Systematic Evolution of Ligands by Exponential Enrichment:

This approach may be employed in an attempt to identify RNA recognition motifs for RNase P RNA (M1 RNA) protein binding for structural analysis as an aid to rational drug design and the secondary evaluation of compounds identified via the HTP screens. The technology is based on the repeated selection and amplification of RNA fragments that specifically bind to a protein with high affinities (Szostak, J. W., TIBS 17: 89-93 (1992)). Fragment libraries based on the S. pneumoniae and E. coli RNase P RNAs may be constructed for the in vitro synthesis of RNA fragments and the subsequent selection of molecules that bind their respective proteins. Chemical and enzymatic structure probing technologies may be employed in combination with protein/RNA protection studies to map the interactive sites. SELEX based on the resulting RNA fragment(s) may be further exploited to determine the minimal structural requirements for RNA recognition.

Disruption of RNase P Assembly

The identification of a protein/RNA-fragment pair permits the development of a screen for compounds that disrupt their assembly. Drug induced disruption of labeled RNA bound to immobilized protein (biotin/streptavidin) would result in the concomitant decrease/loss of the signal generated by the presence of the RNA.

RNA/Drug Interactions

RNase P RNA fragments that confer drug resistance (RNA Fragment Rescue Library supra) may be sequenced and expressed in vitro for chemical and enzymatic structure probing in the presence and absence of the drug in an attempt to map the binding site. SELEX may be applied to lead compounds in an attempt to identify the minimal structural requirements for drug binding.

RNase P Substrates

Minimal RNA substrates may be chemically synthesized for HTP screening including both pre-tRNA and pre-sc RNA derivative. RNA-ligand interactions involving ribose 2′-hydroxyl groups of specific nucleotides may be probed via chemical synthesis of the appropriately modified RNA fragment. In order to retain the C′-endo configuration characteristic of ribonucleotides, 2′-methoxy and 2′-fluororibonucleotides analogues can be used, the latter being preferred on steric grounds. Nucleotides lacking a 2′-substituent adopt the undesired C²-endo configuration.

The invention also relates to inhibitors identified by any of the techniques described herein. Because of the enzymatic nature of RNase P action, it is appreciated that inhibitors may be identified which act as transition state mimics, inhibitors of product release or inhibitors of substrate binding.

Diagnostic Assays

This invention is also related to the use of the RNaseP polynucleotides an polypeptides of the invention for use as diagnostic reagents. Detection of RNaseP in a eukaryote, particularly a mammal, and especially a human, will provide a diagnostic method for diagnosis of a disease. Eukaryotes (herein also “individual(s)”), particularly mammals, and especially humans, infected with an organism comprising the RNaseP gene may be detected at the nucleic acid level by a variety of techniques.

Nucleic acids for diagnosis may be obtained from an infected individual's cells and tissues, such as bone, blood, muscle, cartilage, and skin. Genomic DNA may be used directly for detection or may be amplified enzymatically by using PCR or other amplification technique prior to analysis. RNA or cDNA may also be used in the same ways. Using amplification, characterization of the species and strain of prokaryote present in an individual, may be made by an analysis of the genotype of the prokaryote gene. Deletions and insertions can be detected by a change in size of the amplified product in comparison to the genotype of a reference sequence. Point mutations can be identified by hybridizing amplified DNA to labeled RNaseP polynucleotide sequences. Perfectly matched sequences can be distinguished from mismatched duplexes by RNase digestion or by differences in melting temperatures. DNA sequence differences may also be detected by alterations in the electrophoretic mobility of the DNA fragments in gels, with or without denaturing agents, or by direct DNA sequencing. See, e.g., Myers et al., Science, 230: 1242 (1985). Sequence changes at specific locations also may be revealed by nuclease protection assays, such as RNase and S1 protection or a chemical cleavage method. See, e.g., Cotton et al., Proc. Natl. Acad. Sci., USA, 85: 4397-4401

(1985).

Cells carrying mutations or polymorphisms in the gene of the invention may also be detected at the DNA level by a variety of techniques, to allow for serotyping, for example. For example, RT-PCR can be used to detect mutations. It is particularly preferred to used RT-PCR in conjunction with automated detection systems, such as, for example, GeneScan. RNA or cDNA may also be used for the same purpose, PCR or RT-PCR. As an example, PCR primers complementary to a nucleic acid encoding RNaseP can be used to identify and analyze mutations. Examples of representative primers are shown in the Examples. The invention further provides these primers with 1, 2, 3 or 4 nucleotides removed from the 5′ and/or the 3′ end. These primers may be used for, among other things, amplifying RNaseP DNA isolated from a sample derived from an individual. The primers may be used to amplify the gene isolated from an infected individual such that the gene may then be subject to various techniques for elucidation of the DNA sequence. In this way, mutations in the DNA sequence may be detected and used to diagnose infection and to serotype and/or classify the infectious agent.

The invention further provides a process for diagnosing, disease, preferably bacterial infections, more preferably infections by S. pneumoniae, and most preferably disease, such as, otitis media, conjunctivitis, pneumonia, bacteremia, meningitis, sinusitis, pleural empyema and endocarditis, and most particularly meningitis, such as for example infection of cerebrospinal fluid, comprising determining from a sample derived from an individual a increased level of expression of polynucleotide having the sequence of Table 1 [any of SEQ ID NO: 1, 3, 4, 5 or 7]. Increased or decreased expression of RNaseP polynucleotide can be measured using any on of the methods well known in the art for the quantitation of polynucleotides, such as, for example, amplification, PCR, RT-PCR, RNase protection, Northern blotting and other hybridization methods.

In addition, a diagnostic assay in accordance with the invention for detecting over-expression of RNaseP protein compared to normal control tissue samples may be used to detect the presence of an infection, for example. Assay techniques that can be used to determine levels of a RNaseP protein, in a sample derived from a host are well-known to those of skill in the art. Such assay methods include radioimmunoassays, competitive-binding assays, Western Blot analysis and ELISA assays.

Antibodies

The polypeptides of the invention or variants thereof, or cells expressing them can be used as an immunogen to produce antibodies immunospecific for such polypeptides. “Antibodies” as used herein includes monoclonal and polyclonal antibodies, chimeric, single chain, simianized antibodies and humanized antibodies, as well as Fab fragments, including the products of an Fab immunolglobulin expression library.

The Fab fragment may also be prepared from its parent monoclonal antibody by enzyme treatment, for example using papain to cleave the Fab portion from the Fc portion.

Antibodies generated against the polypeptides of the invention can be obtained by administering the polypeptides or epitope-bearing fragments, analogues or cells to an animal, preferably a nonhuman, using routine protocols. The antibody so obtained will then bind the polypeptides itself. In this manner, even a sequence encoding only a fragment of the polypeptides can be used to generate antibodies binding the whole native polypeptides. Such antibodies can then be used to isolate the polypeptide from tissue expressing that polypeptide. For preparation of monoclonal antibodies, any technique known in the art that provides antibodies produced by continuous cell line cultures can be used. Examples include various techniques, such as those in Kohler, G. and Milstein, C., Nature 256: 495-497 (1975); Kozbor et al., Immunology Today 4: 72 (1983); Cole et al., pg. 77-96 in MONOCLONAL ANTIBODIES AND CANCER THERAPY, Alan R. Liss, Inc. (1985), )), and the EBV-hybridoma technique to produce human monoclonal antibodies (Cole, et al., 1985, in Monoclonal Antibodies and Cancer Therapy, Alan R. Liss, Inc., pp. 77-96).

The hybridomas are screened to select a cell line with high binding affinity and favorable cross reaction with other staphylococcal species using one or more of the original polypeptide and/or the fusion protein. The selected cell line is cultured to obtain the desired Mab.

Techniques for the production of single chain antibodies (U.S. Pat. No. 4,946,778) can be adapted to produce single chain antibodies to polypeptides of this invention. Also, transgenic mice, or other organisms such as other mammals, may be used to express humanized antibodies.

Alternatively phage display technology may be utilized to select antibody genes with binding activities towards the polypeptide either from repertoires of PCR amplified v-genes of lymphocytes from humans screened for possessing anti-RNaseP or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

The antibody should be screened again for high affinity to the polypeptide and/or fusion protein.

As mentioned above, a fragment of the final antibody may be prepared.

The antibody may be either intact antibody of Mᵣ approx 150,000 or a derivative of it, for example a Fab fragment or a Fv fragment as described in Skerra, A and Pluckthun, A., Science 240:1038-1040 (1988). If two antigen binding domains are present each domain may be directed against a different epitope—termed ‘bispecific’ antibodies.

If two antigen binding domains are present each domain may be directed against a different epitope—termed bispecific antibodies.

In particular derivatives which are slightly longer or slightly shorter than the native protein or polypeptide fragment of the present invention may be used. In addition, polypeptides in which one or more of the amino acid residues are modified may be used. Such peptides may, for example, be prepared by substitution, addition, or rearrangement of amino acids or by chemical modification thereof. All such substitutions and modifications are generally well known to those skilled in the art of peptide chemistry.

The above-described antibodies may be employed to isolate or to identify clones expressing the polypeptides to purify the polypeptides by affinity chromatography.

Thus, among others, antibodies against RNaseP polypeptide or polynucleotide, including for example RNase P RNA, may be employed to treat infections, particularly bacterial infections and especially disease, such as, otitis media, conjunctivitis, pneumonia, bacteremia, meningitis, sinusitis, pleural empyema and endocarditis, and most particularly meningitis, such as for example infection of cerebrospinal fluid.

Preferably the antibody is prepared by expression of a DNA polymer encoding said antibody in an appropriate expression system such as described above for the expression of polypeptides of the invention. The choice of vector for the expression system will be determined in part by the host, which may be a prokaryotic cell, such as E. coli (preferably strain B) or Streptomyces sp. or a eukaryotic cell, such as a mouse C127, mouse myeloma, human HeLa, Chinese hamster ovary, filamentous or unicellular fungi or insect cell. The host may also be a transgenic animal or a transgenic plant (for example, as described in Hiatt, A. et al., Nature 340:76-78(1989). Suitable vectors include plasmids, bacteriophages, cosmids and recombinant viruses, derived from, for example, baculoviruses and vaccinia.

Polypeptide variants include antigenically, epitopically or immunologically equivalent variants that form a particular aspect of this invention. The term “antigenically equivalent derivative” as used herein encompasses a polypeptide or its equivalent which will be specifically recognized by certain antibodies which, when raised to the protein or polypeptide according to the invention, interfere with the immediate physical interaction between pathogen and mammalian host. The term “immunologically equivalent derivative” as used herein encompasses a peptide or its equivalent which when used in a suitable formulation to raise antibodies in a vertebrate, the antibodies act to interfere with the immediate physical interaction between pathogen and mammalian host.

The polypeptide, such as an antigenically or immunologically equivalent derivative or a fusion protein thereof is used as an antigen to immunize a mouse or other animal such as a rat or chicken. The fusion protein may provide stability to the polypeptide. The antigen may be associated, for example by conjugation, with an immunogenic carrier protein for example bovine serum albumin (BSA) or keyhole limpet haemocyanin (KLH). Alternatively a multiple antigenic peptide comprising multiple copies of the protein or polypeptide, or an antigenically or immunologically equivalent polypeptide thereof may be sufficiently antigenic to improve immunogenicity so as to obviate the use of a carrier.

Preferably, the antibody or variant thereof is modified to make it less immunogenic in the individual. For example, if the individual is human the antibody may most preferably be “humanized”; where the complimentarity determining region(s) of the hybridoma-derived antibody has been transplanted into a human monoclonal antibody, for example as described in Jones, P. et al. (1986), Nature 321, 522-525 or Tempest et al., (1991) Biotechnology 9, 266-273. The humanized monoclonal antibody, or its fragment having binding activity, form a particular aspect of this invention.

The modification need not be restricted to one of “humanization”; other primate sequences (for example Newman, R. et al., Biotechnology 10:1455-1460 (1992)) may also be used.

The use of a polynucleotide of the invention in genetic immunization will preferably employ a suitable delivery method such as direct injection of plasmid DNA into muscles (Wolff et al., Hum Mol Genet 1992, 1:363, Manthorpe et al., Hum. Gene Ther. 1963:4, 419), delivery of DNA complexed with specific protein carriers (Wu et al., J. Biol Chem. 1989: 264,16985), coprecipitation of DNA with calcium phosphate (Benvenisty & Reshef, PNAS USA, 1986:83,9551), encapsulation of DNA in various forms of liposomes (Kaneda et al., Science 1989:243,375), particle bombardment (Tang et al., Nature 1992, 356:152, Eisenbraun et al., DNA Cell Biol 1993, 12:79 1) and in vivo infection using cloned retroviral vectors (Seeger et al., PNAS USA 1984:81,5849).

Vaccines

Another aspect of the invention relates to a method for inducing an immunological response in an individual, particularly a mammal which comprises inoculating the individual with RNaseP, or a fragment or variant thereof, adequate to produce antibody and/ or T cell immune response to protect said individual from infection, particularly bacterial infection and most particularly S. pneumoniae infection. Also provided are methods whereby such immunological response slows bacterial replication. Yet another aspect of the invention relates to a method of inducing immunological response in an individual which comprises delivering to such individual a nucleic acid vector to direct expression of RNaseP, or a fragment or a variant thereof, for expressing RNaseP, or a fragment or a variant thereof in vivo in order to induce an immunological response, such as, to produce antibody and/or T cell immune response, including, for example, cytokine-producing T cells or cytotoxic T cells, to protect said individual from disease, whether that disease is already established within the individual or not. One way of administering the gene is by accelerating it into the desired cells as a coating on particles or otherwise. Such nucleic acid vector may comprise DNA, RNA, a modified nucleic acid, or a DNA/RNA hybrid.

A further aspect of the invention relates to an immunological composition which, when introduced into an individual capable or having induced within it an immunological response, induces an immunological response in such individual to a RNaseP or protein coded therefrom, wherein the composition comprises a recombinant RNaseP or protein coded therefrom comprising DNA which codes for and expresses an antigen of said RNaseP or protein coded therefrom. The immunological response may be used therapeutically or prophylactically and may take the form of antibody immunity or cellular immunity such as that arising from CTL or CD4+ T cells.

A RNaseP polypeptide or a fragment thereof may be fused with co-protein which may not by itself produce antibodies, but is capable of stabilizing the first protein and producing a fused protein which will have immunogenic and protective properties. Thus fused recombinant protein, preferably further comprises an antigenic co-protein, such as lipoprotein D from Hemophilus influenizae, Glutathione-S-transferase (GST) or beta-galactosidase, relatively large co-proteins which solubilize the protein and facilitate production and purification thereof. Moreover, the co-protein may act as an adjuvant in the sense of providing a generalized stimulation of the immune system. The co-protein may be attached to either the amino or carboxy terminus of the first protein.

An RNaseP polynucleotide, particularly Rnase P RNA, or a fragment thereof may be fused with RNA, DNA or PNA (herein such fusion is referred to as “co-polynucleotide”) and this co-polynucleotide may not by itself produce antibodies, but is capable of stabilizing the first polynucleotide and producing a fused polynucleotide which will have immunogenic and protective properties. Thus fused recombinant polynucleotide, preferably further comprises an antigenic co-polynucleotide or a polynucleotide that will aid in purification. Moreover, the co-polynucleotide may act as an adjuvant in the sense of providing a generalized stimulation of the immune system. The co-polynucleotide may be attached to either the 5′ or 3′ terminus of the first polynucleotide.

Provided by this invention are compositions, particularly vaccine compositions, and methods comprising the polypeptides or polynucleotides of the invention and immunostimulatory DNA sequences, such as those described in Sato, Y. et al. Science 273: 352 (1996).

Also, provided by this invention are methods using the described polynucleotide or particular fragments thereof which have been shown to encode non-variable regions of bacterial cell surface proteins in DNA constructs used in such genetic immunization experiments in animal models of infection with S. pneumoniae will be particularly useful for identifying protein epitopes able to provoke a prophylactic or therapeutic immune response. It is believed that this approach will allow for the subsequent preparation of monoclonal antibodies of particular value from the requisite organ of the animal successfully resisting or clearing infection for the development of prophylactic agents or therapeutic treatments of bacterial infection, particularly S. pneumoniae infection, in mammals, particularly humans.

The polypeptide may be used as an antigen for vaccination of a host to produce specific antibodies which protect against invasion of bacteria, for example by blocking adherence of bacteria to damaged tissue. Examples of tissue damage include wounds in skin or connective tissue caused, e.g., by mechanical, chemical or thermal damage or by implantation of indwelling devices, or wounds in the mucous membranes, such as the mouth, mammary glands, urethra or vagina.

The invention also includes a vaccine formulation which comprises an immunogenic recombinant protein of the invention together with a suitable carrier. Since the protein may be broken down in the stomach, it is preferably administered parenterally, including, for example, administration that is subcutaneous, intramuscular, intravenous, or intradermal. Formulations suitable for parenteral administration include aqueous and non-aqueous sterile injection solutions which may contain anti-oxidants, buffers, bacteriostats and solutes which render the formulation insotonic with the bodily fluid, preferably the blood, of the individual; and aqueous and non-aqueous sterile suspensions which may include suspending agents or thickening agents. The formulations may be presented in unit-dose or multi-dose containers, for example, sealed ampules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

While the invention has been described with reference to certain RNaseP protein, it is to be understood that this covers fragments of the naturally occurring protein and similar proteins with additions, deletions or substitutions which do not substantially affect the immunogenic properties of the recombinant protein.

Compositions, kits and administration

The invention also relates to compositions comprising the polynucleotide or the polypeptides discussed above or their agonists or antagonists. The polypeptides of the invention may be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of a polypeptide of the invention and a pharmaceutically acceptable carrier or excipient. Such carriers may include, but are not limited to, saline, buffered saline, dextrose, water, glycerol, ethanol and combinations thereof. The formulation should suit the mode of administration. The invention further relates to diagnostic and pharmaceutical packs and kits comprising one or more containers filled with one or more of the ingredients of the aforementioned compositions of the invention.

Polypeptides and other compounds of the invention may be employed alone or in conjunction with other compounds, such as therapeutic compounds.

The pharmaceutical compositions may be administered in any effective, convenient manner including, for instance, administration by topical, oral, anal, vaginal, intravenous, intraperitoneal, intramuscular, subcutaneous, intranasal or intradermal routes among others.

In therapy or as a prophylactic, the active agent may be administered to an individual as an injectable composition, for example as a sterile aqueous dispersion, preferably isotonic.

Alternatively the composition may be formulated for topical application for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

For administration to mammals, and particularly humans, it is expected that the daily dosage level of the active agent will be from 0.01 mg/kg to 10 mg/kg, typically around 1 mg/kg. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

In-dwelling devices include surgical implants, prosthetic devices and catheters, i.e., devices that are introduced to the body of an individual and remain in position for an extended time. Such devices include, for example, artificial joints, heart valves, pacemakers, vascular grafts, vascular catheters, cerebrospinal fluid shunts, urinary catheters, continuous ambulatory peritoneal dialysis (CAPD) catheters.

The composition of the invention may be administered by injection to achieve a systemic effect against relevant bacteria shortly before insertion of an in-dwelling device. Treatment may be continued after surgery during the in-body time of the device. In addition, the composition could also be used to broaden perioperative cover for any surgical technique to prevent bacterial wound infections, especially S. pneumoniae wound infections.

Many orthopaedic surgeons consider that humans with prosthetic joints should be considered for antibiotic prophylaxis before dental treatment that could produce a bacteremia. Late deep infection is a serious complication sometimes leading to loss of the prosthetic joint and is accompanied by significant morbidity and mortality. It may therefore be possible to extend the use of the active agent as a replacement for prophylactic antibiotics in this situation.

In addition to the therapy described above, the compositions of this invention may be used generally as a wound treatment agent to prevent adhesion of bacteria to matrix proteins exposed in wound tissue and for prophylactic use in dental treatment as an alternative to, or in conjunction with, antibiotic prophylaxis. Alternatively, the composition of the invention may be used to bathe an in-dwelling device immediately before insertion. The active agent will preferably be present at a concentration of 1 μg/ml to 10 mg/ml for bathing of wounds or indwelling devices.

A vaccine composition is conveniently in injectable form. Conventional adjuvants may be employed to enhance the immune response. A suitable unit dose for vaccination is 0.5-5 microgram/kg of antigen, and such dose is preferably administered 1-3 times and with an interval of 1-3 weeks. With the indicated dose range, no adverse toxicological effects will be observed with the compounds of the invention which would preclude their administration to suitable individuals.

Each reference disclosed herein is incorporated by reference herein in its entirety. Any patent application to which this application claims priority is also incorporated by reference herein in its entirety.

### EXAMPLES

The examples below are carried out using standard techniques, which are well known and routine to those of skill in the art, except where otherwise described in detail. The examples are illustrative, but do not limit the invention.

### Example 1

An example of an assay to affect and detect Strep. pneumo. RNase P cleavage is as follows. This assay will first require a kinetic analysis to obtain an appropriate K_{M} and k_{cat}, to screen. For Strep. Pneumoniae, one simply would prefer to see activity. So one may use the Streptococcus pneumoniae RNA and either the E. coli or Staphylococcus aureus protein to form the holoenzyme, if formation of the holoenzyme is desired for the particular assay. In order to readily measure catalytic activity, the holoenzymes are are preferably prepared to a final concentration of 100 nM in the assay and the ³²P-substrate analog was present in only trace amounts (for example, amounts of about 50,000 counts per minute (CPM) per reaction).

The RNAs (including the ³²p-substrate analog) were added to dH₂O and denatured at 95° C. for 1.5 minutes. They were then allowed to cool for approximately 3-4 minutes at room temperature. At this point, 2×buffer (200 mM Tris pH 7.0, 300 mM KCl, 20 mM MgCl, 10% PEG) was added to the RNA/dH₂O mixtures and incubated for 4-5 minutes at room temperature. Now, the respective proteins are gently mixed in with the RNA/dH₂O/2×buff.

The substrate gets no protein. All cocktails continue to incubate at room temperature for another 10 minutes. After this incubation, the reaction is initiated by adding the holoenzyme cocktail to the substrate cocktail and incubated at room temperature for 30 minutes. The reactions are stopped by adding a volume 1:1 ratio of quench buffer to the reaction (usually 10 microliter reaction is run when screening ). At this point, the reactions were loaded and run on a gel to visualize and quantitate the products of the reaction.

The E. coli C5 RNaseP protein may be used in the reaction described in this example as well as in other screening and activity assays set forth elsewhere herein. This protein is disclosed in, for example, Hansen F., E. Hansen. and T. Atlung. 1985. Physical mapping and nucleotide sequence of the mpA gene that encodes the protein component of ribonuclease P in Escherichia coli. Gene 38:85-93.

What is claimed is:

1. An isolated polynucleotide comprising a polynucleotide sequence selected from the group consisting of:
(a) a polynucleotide having at least a 50% identity to a polynucleotide comprising the nucleic acid sequence of SEQ ID NO:3, 4 or 7;
(b) a polynucleotide having at least a 50% identity to a polynucleotide comprising the same catalytic RNA expressed by the RNaseP RNA gene contained in the S. pneumoniae of the deposited strain;
(c) a polynucleotide which is complementary to the polynucleotide of (a), (b), or (c); and
(d) a polynucleotide comprising at least 15 sequential bases of the polynucleotide of (a), (b), or (c).

2. The polynucleotide of claim 1 wherein the polynucleotide is DNA.

3. The polynucleotide of claim 1 wherein the polynucleotide is RNA.

4. A polynucleotide isolated from Streptococcus pneumoniae selected from the group consisting of:
(a) a polynucleotide of at least 30 nucleotides in length obtained by hybridizing with the nucleic acid sequence set forth in SEQ ID NO:1, 3, 4, 5 or 7 as a probe or primer, and
(b) the polynucleotide set forth in SEQ ID NO: 1, 3, 4, 5 or 7
(a) a polynucleotide fully complementary to the sequence of a polynucleotide of (a) or (b).

5. The polynucleotide of claim 2 comprising nucleotides set forth in SEQ ID NO:3, 4 or 7.

6. A vector comprising the polynucleotide of claim 1.

7. A host cell comprising the vector of claim 6.

8. A process for producing an RNA comprising: expressing from the host cell of claim 7 an RNA transcribed by said DNA.

9. A process for producing a RNaseP RNA or fragment comprising culturing a host of claim 7 under conditions sufficient for the production of said RNA or fragment.

10. The polynucleotide of claim 3 wherein said RNA is a catalytic RNA.

11. An antagonist which inhibits the activity or expression of the RNA of claim 3.

12. A method for the treatment of an individual having need to inhibit RNaseP RNA comprising: administering to the individual a therapeutically effective amount of the antagonist of claim 11.

13. A process for diagnosing a disease related to expression or activity of an RNA of claim 3 in an individual comprising:
(a) determining a nucleic acid sequence transcribing said RNA, and/or
(b) analyzing for the presence or amount of said RNA in a sample derived from the individual.

14. A method for identifying compounds which interact with and inhibit or activate an activity of the RNA of claim 10 comprising:
contacting a composition comprising the RNA with the compound to be screened under conditions to permit interaction between the compound and the RNA to assess the interaction of a compound with such RNA, such interaction being associated with a second component capable of providing a detectable signal in response to the interaction of the polypeptide with the compound;
and determining whether the compound interacts with and activates or inhibits an activity of the RNA by detecting the presence or absence of a signal generated from the interaction of the compound with the RNA.

15. An isolated RNase P from S. pneumoniae.

16. The RNase P according to claim 15 having an RNA component of the sequence of SEQ ID NO: 3.

17. An isolated RNA component of S. pneumoniae RNase P.

18. An isolated protein component of S. pneumoniae RNase P.

19. An isolated DNA encoding the components of claim 18.

30. A polynucleotide comprising a polynucleotide sequence selected from the group consisting of SEQ ID NO:3, 4, 5 or 7.

31. A polynucleotide consisting of a polynucleotide sequence selected from the group consisting of SEQ ID NO: 3, 4, 5 or 7.
